SEQUENCE LISTING (11 sequences)

SEQ ID NO: 1 - DNA, 1110 bp, Homo sapiens
  CDS (66)..(737)
cctgggcggctccgctagctgtttttcgtcttccctaggctatttctgccgggcgctccgcgaagatgcagctcaagccgatggagatcaaccccgagatgctgaacaaagtgctgtcccggctgggggtcgccggccagtggcgcttcgtggacgtgctggggctggaagaggagtctctgggctcggtgccagcgcctgcctgcgcgctgctgctgctgtttcccctcacggcccagcatgagaacttcaggaaaaagcagattgaagagctgaagggacaagaagttagtcctaaagtgtacttcatgaagcagaccattgggaattcctgtggcacaatcggacttattcacgcagtggccaataatcaagacaaactgggatttgaggatggatcagttctgaaacagtttctttctgaaacagagaaaatgtcccctgaagacagagcaaaatgctttgaaaagaatgaggccatacaggcagcccatgatgccgtggcacaggaaggccaatgtcgggtagatgacaaggtgaatttccattttattctgtttaacaacgtggatggccacctctatgaacttgatggacgaatgccttttccggtgaaccatggcgccagttcagaggacaccctgctgaaggacgctgccaaggtctgcagagaattcaccgagcgtgagcaaggagaagtccgcttctctgccgtggctctctgcaaggcagcctaatgctctgtgggagggactttgctgatttcccctcttcccttcaacatgaaaatatatacccccccatgcagtctaaaatgcttcagtacttgtgaaacacagctgttcttctgttctgcagacacgccttcccctcagccacacccaggcacttaagcacaagcagagtgcacagctgtccactgggccattgtggtgtgagcttcagatggtgaagcattctccccagtgtatgtcttgtatccgatatctaacgctttaaatggctactttggtttctgtctgtaagttaagaccttggatgtggtttaattgtttgtcctcaaaaggaataaaacttttctgctgataagataaaaaaaaaaaaaaaaaa

SEQ ID NO: 2 - protein, 223 aa, Homo sapiens [sequence omitted]

SEQ ID NO: 3 - DNA, 995 bp, Mus musculus
  CDS (9)..(680)
ctaaagagatgcagctgaagccgatggagattaaccccgagatgctgaacaaagtgttggccaagctgggggtcgccggccagtggcgcttcgccgacgtgctagggctggaggaggagactctgggctcagtgccatcccctgcctgcgccctgctgctcctgtttcccctcacggcccagcatgaaaacttcaggaaaaagcaaattgaggaactgaagggacaggaagttagccctaaagtttacttcatgaagcagaccatcggaaactcctgtggtaccatcgggttgatccacgcagtggccaacaaccaagacaagctggaatttgaggatggatccgtcctgaaacagtttctgtctgaaacggagaagctgtcccccgaagatagagccaagtgtttcgagaagaacgaggccatccaggcagcccatgactccgtggcccagaagggccagtgtcgggtagatgacaaagtgaatttccattttattctgttcaacaacgtggacggccatctgtacgagctcgatgggcgaatgccctttccagtgaaccatggcgccagctcagaggactctctgctgcaggatgctgccaaggtctgcagagaattcactgagcgcgagcagggggaggtccgcttctctgccgtggctctctgcaaagcagcttaagtctggggagagagaaccagccgatccccccttccctgggcaggtgcgcgcggccccgcccttggtttgcagctttagcacttagaaccacagctgtcttcttgcgttctacagccccatcccctccaccccacccaggccaccagggggctctgtcacagccacaccaggctgagcactttccctcctgtgtgtctcgtaccttgctctctacggtctctttggtttctgtctgtaagttatggccctggatgggtttgtctagtccttaaaaggaagaataaaactttgctggtgagagtatcaaaaaaaaaa

SEQ ID NO: 4 - protein, 223 aa, Mus musculus
MQLKPMEINPEMLNKVLAKLGVAGQWRFADVLGLEEETLGSVPSPACALLLLFPLTAQHENFRKKQIEELKGQEVSPKVYFMKQTIGNSCGTIGLIHAVANNQDKLEFEDGSVLKQFLSETEKLSPEDRAKCFEKNEAIQAAHDSVAQKGQCRVDDKVNFHFILFNNVDGHLYELDGRMPFPVNHGASSEDSLLQDAAKVCREFTEREQGEVRFSAVALCKAA

SEQ ID NO: 5 - DNA, 30 bp, Artificial sequence
  synthetic oligonucleotide
ctaaagcttatgcagctcaagccgatggag

SEQ ID NO: 6 - DNA, 33 bp, Artificial sequence
  synthetic oligonucleotide
ctaagactcgagttaggctgccttgctgagagc

SEQ ID NO: 7 - DNA, 58 bp, Artificial sequence
  synthetic oligonucleotide
ctaaagcttatggactacaaggatgacgacgacaaagatgcagctcaagccgatggag

SEQ ID NO: 8 - DNA, 30 bp, Artificial sequence
  synthetic oligonucleotide
atcctcgagttaggctgccttgacgagagc

SEQ ID NO: 9 - DNA, 58 bp, Artificial sequence
  synthetic oligonucleotide
ctaaagcttatggactacaaggatgacgacgacaaagatggagggtcaacgctggctg

SEQ ID NO: 10 - DNA, 34 bp, Artificial sequence
  synthetic oligonucleotide
atcctcgagctatgctgcagaaagagcaatcgca

SEQ ID NO: 11 - DNA, 44 bp, Artificial sequence
  synthetic oligonucleotide
atcctcgagctatgctgccttagaaagagcaatcgcattaaatc

(19) United States
(12) Patent Application Publication
(10) Pub. No.: US 2005/0272068 A1
(43) Pub. Date: Dec. 8, 2005

(54) UCH-L1 expression and cancer therapy

(75) Inventors: Peter T. Lansbury, Brookline, MA (US); Yichin Liu, Palo Alto, CA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: The Brigham and Women's Hospital, Inc., Boston, MA (US)

(21) Appl. No.: 11/084,716

(22) Filed: Mar. 18, 2005

Related U.S. Application Data
(60) Provisional application No. 60/554,634, filed on Mar. 18, 2004.

(51) Int. Cl. C12Q 1/68; G01N 33/574

(52) U.S. Cl. 435/6; 435/7.23

(57) ABSTRACT

Methods are provided of treating cancer with one or more farnesyl transferase inhibitors. Methods are provided for identifying cancers that are particularly susceptible to treatment with one or more farneseyl transferase inhibitors by identifying cancers that express low levels of UCH-L1.

### RELATED APPLICATIONS

This application claims benefit under 35 U.S.C. 119(e) of the filing date of U.S. Ser. No. 60/554,634 filed on Mar. 18, 2004, the entire disclosure of which is incorporated herein by reference.

### FEDERALLY SPONSORED RESEARCH

This invention was made with Government support under NIH (National Institute of Health) Grant No. NS38375. The Government may have certain rights to this invention.

### FIELD OF THE INVENTION

The present invention relates to methods for identifying appropriate drug regimens for treating certain cancers. The invention also relates to therapeutic approaches for the treatment of cancer.

### BACKGROUND OF THE INVENTION

Typical treatment regimens for breast cancer involve endocrine-based drugs (oestrogen receptor antagonists, aromatase inhibitors etc.) and conventional cytotoxics (doxorubicin, cyclophosphamide, 5-fluorouracil etc.). In addition, certain signal transduction inhibitor drugs have been suggested for treating breast cancer. For example, farnesyl transferase inhibitors (FTIs) are being tested clinically as treatment options for breast cancer. However, like many cancer therapies, FTIs are not uniformally effective against all breast cancers. In addition, FTIs are not always effective when tested on cancers other than breast cancer.

There is therefore a need in the art for methods and compositions for enhancing the anti-cancer properties of FTIs.

### SUMMARY OF THE INVENTION

Aspects of the present invention relate to methods for identifying cancers that are responsive to treatment with farnesyl transferase inhibitor compounds. The invention also provides methods and compositions for treating cancer. Applicants have discovered that certain cancers express higher levels of UCH-L1 than other cancers, that the expression of UCH-L1 can have an anti-proliferative effect on cancer cell growth, and that a farnesyl transferase inhibitor can reduce the anti-proliferative effect of UCH-L1. Therefore, the anti-cancer effects of a farnesyl transferase inhibitor (e.g., due to its action on certain signal transduction pathways) may be countered by its inhibition of UCH-L1 activity in cancers that express UCH-L1 (e.g., over-express UCH-L1 or express UCH-L1 at levels above a threshold level). According to the invention, this explains the inconsistent results observed with farnesyl transferase inhibitor treatment of cancer and suggests that farnesyl transferase inhibitor treatment may be more effective on cancers that don't express UCH-L1 or express low levels of UCH-L1 (e.g., levels below a threshold or reference level).

Accordingly, one aspect of the invention provides a screening method for identifying cancers that are responsive to treatment with one or more farnesyl transferase inhibitors. In one embodiment, a screening method may include detecting an amount of UCH-L1 expression in a cancer sample (e.g., a biological sample containing cancer cells, for example a tissue biopsy or a sample of biological fluid or excretia), and identifying the cancer as either (i) a farnesyl transferase responsive cancer if the amount of UCH-L1 is below a threshold amount; or (ii) a farnesyl transferase non-responsive cancer if the amount of UCH-L 1 is above the threshold amount. The threshold amount of UCH-L1 may be an amount of UCH-L1 expressed in a reference cell line, an amount expressed in a reference cancer, and/or an amount that is above that of normal tissue (e.g., between about 1.5 and about 10 times or more than that in normal tissue, for example about two times or more than that in normal tissue). The amount of UCH-L1 expression may be detected using an RNA expression assay, a protein expression assay, an ELISA, a UCH-L1 activity assay, and/or any other suitable assay. The amount of expression may be measured in a biological sample, for example a tissue biopsy, cell culture, a bodily fluid, and/or any other suitable biological sample.

In another aspect, the invention provides a method for treating cancer by identifying a cancer with a UCH-L1 expression amount that is below a threshold amount; and treating a subject having such a cancer with a therapeutically effective amount of a farnesyl transferase inhibitor.

In another aspect, the invention provides a screening method to identify cancers responsive to farnesyl transferase inhibitors by detecting an amount of UCH-L1 expression in a cancer in a patient in vivo; and identifying the cancer as either (i) a farnesyl transferase responsive cancer if the amount of UCH-L1 is below a threshold amount, or (ii) a farnesyl transferase non-responsive cancer if the amount of UCH-L1 is above the threshold amount.

In yet another aspect, the invention provides a method of treating cancer by identifying a cancer with a UCH-L1 expression amount above a threshold amount; and treating a subject with the cancer with a therapy that is not a farnesyl transferase inhibitor-based therapy. The therapy may include surgery. The therapy may include endocrine-based drug therapy.

In a further embodiment, the invention provides a method for treating a cancer patient by administering a composition comprising a farnesyl transferase inhibitor to a patient with a UCH-L1 level below a threshold level. In one embodiment, the patient is known to have a cancer with a UCH-L1 level below a threshold level. In one embodiment, the patient may be tested and identified as having a cancer with a UCH-L1 level below a threshold level.

It should be appreciated that in any of the aspects and embodiments described herein, the subject may be a human subject (e.g., a human cancer patient). Furthermore, the farnesyl transferase inhibitor may be SCH66336, L778123, L744832, BMS-214662, R115777, FTI-1, FTI-2 and/or FTI-277. In certain embodiments, two or more different farnesyl transferase inhibitors may be administered (e.g., between 2 and 50, between 2 and 25, between 2 and 10, 2, 3, 4, 5, 6, 7, 8, or 9). For example two or more of the following farnesyl transferase inhibitors may be administered: SCH66336 (FIG. 9A), SCH 44342 (FIG. 9B), L778123 (FIG. 10), L744832 (FIG. 11), BMS-214662 (FIG. 8), R115777 (FIG. 7), FTI-1 (FIG. 11), FTI-2 (FIG. 11) and FTI-277. In addition, the farnesyl transferase inhibitor(s) may be administered in combination with one or more different anticancer drugs.

It should be appreciated that aspects and embodiments of the invention described herein in connection with one farnesyl transferase inhibitor also may be practiced using two or more farnesyl transferase inhibitors (e.g., between 2 and 50, between 2 and 25, between 2 and 10, 2, 3, 4, 5, 6, 7, 8, or 9). Similarly, aspects and embodiments of the invention described herein in connection with one other compound also may be practiced using two or more other compounds (e.g., between 2 and 50, between 2 and 25, between 2 and 10, 2, 3, 4, 5, 6, 7, 8, or 9).

### BRIEF DESCRIPTION OF THE DRAWINGS

FIG. 1 shows that UCH-L1 membrane association is regulated by its farnesylation.

FIG. 2 shows that C220S mutation abolished the inhibitory effect of UCH-L1 WT on α-synuclein degradation.

FIG. 3 shows that farnesyl transferase inhibitor can rescue the α-synuclein toxicity in infected SH—SY5Y cells.

FIG. 4 shows that FTI-277 rescued α-synuclein toxicity in SH—SY5Y cells by reducing the amount of α-synuclein accumulation.

FIG. 5 shows that the anti-proliferative effect of UCH-L1 is mediated by its farnesylation.

FIG. 6 shows that UCH-L1 inhibition in H1299 reduces the amount of p27: (A) the chemical structure of UCH-L1 inhibitor (LDN-57414); (B) the amount of p27 in H1299 treated with either DMSO or LDN-57414 was analyzed by Western blot.

FIG. 7 shows the structure of compound R115777.

FIG. 8 shows the structure of compound BMS 214662.

FIG. 9 shows the structure of compound SCH 66336 (9A) and SCH 44342 (9B).

FIG. 10 shows the structure of compound L-778,123.

FIG. 11 shows the structures of compounds FTI-1, FTI-2, and L-744832.

### DETAILED DESCRIPTION

The invention provides methods, compositions and articles of manufacture for treating diseases associated with uncontrolled cell proliferation (e.g., tumors and cancers). The invention is based, in part, on the discovery that the effectiveness of farnesyl transferase inhibitors (FTIs) in cancer treatment is dependent on the expression level of the enzyme UCH-L1 in the cancer cells or tissue. UCH-L1 (ubiquitin C-terminal hydrolase L1) is involved in ubiquitin-based protein degradation pathways. According to the invention, cancer cells are responsive to FTI-based therapies when UCH-L1 levels are below a threshold level. In contrast, cancer cells are minimally responsive to FTI-based therapies when UCH-L1 levels are above a threshold level.

In one aspect, the invention is useful for identifying cancers that are responsive to FTI treatment. In some embodiments, the level of UCH-L1 expression in a cancer is assayed and the UCH-L1 expression level is analyzed to determine the responsiveness of the cancer to an FTI. For example, the expression level can be compared to one or more reference or threshold levels to determine the responsiveness of the cancer.

In another aspect, the invention is useful to screen a population of cancer patients to identify those that should be treated with an FTI. The invention is also useful to identify patients that should be treated with a drug or therapy regimen that does not include an FTI inhibitor.

In another aspect, the invention provides a combination therapy that includes an FTI inhibitor and a UCH-L1 inhibitor. According to the invention, this combination therapy is particularly useful to treat cancers that express UCH-L1. However, this combination therapy may also be appropriate for cancers that do not express UCH-L1 to prevent the outgrowth and/or metastasis of one or more cells that don't express UCH-L1.

In another aspect, the invention provides methods for identifying patients that should be treated with a combination of an FTI and a UCH-L1 inhibitor. In some embodiments, the level of UCH-L1 expression in a cancer is determined, and a patient is identified as a candidate for a combination therapy if the UCH-L1 expression level is above one or more reference or threshold levels.

Accordingly, in some embodiments, the invention provides methods for treating a cancer patient including the step of administering to the patient a therapeutically effective amount of a farnesyl transferase inhibitor compound or a therapeutical preparation of the compound. In other embodiments, the invention provides methods for treating a cancer patient including the step of administering to the patient a therapeutically effective amount of a farnesyl transferase inhibitor compound and a therapeutically effective amount of a UCH-L1 inhibitor compound or a therapeutical preparation of these compounds. The farnesyl transferase inhibitor compound can be administered along with, before, or after the UCH-L1 inhibitor compound. In some embodiments, the farnesyl transferase inhibitor compound and UCH-L1 inhibitor compounds can be formulated into a single therapeutic preparation. These different inhibitor compounds and preparations described herein can be administered as a single dose or in several doses administered over a period of time (e.g., chronic administration at regular intervals of time) as described herein. Methods and compositions of the invention include stereoisomeric forms and pharmaceutically acceptable acid or base addition salt forms of the farnesyl transferase inhibitors and UCH-L1 inhibitors.

UCH-L1

Ubiquitin C-terminal hydrolase-1 (UCH-L1) is both a ubiquityl hydrolase and a ubiquityl ligase. It is expressed in the brain, testis, ovaries, and in some tumors, where it has antiproliferative activity. A UCH-L1 polymorphism that protects against Parkinson's disease reduces its ligase activity in vitro and reduces α-synuclein accumulation in cultured cells. According to the invention, membrane-associated UCH-L1 is farnesylated in vivo. Elimination of the farnesylation site does not affect the in vitro enzymatic activities, but eliminates the ability of UCH-L1 to promote accumulation of α-synuclein in cells and its ability to inhibit proliferation of a lung cancer cell line. These effects suggest that both effects are mediated by dimer-dependent ligase activity. Inhibition of farnesylation with the drug-like molecule FTI-277 protects neuroblastoma cells from α-synuclein toxicity. The effect of FTI-277 on lung cancer cells depends on whether they express UCH-L1: it is antiproliferative only if UCH-L1 is not expressed. The association of UCH-L1 farnesylation with neuroprotection on the one hand and antiproliferative activity on the other has significant implications for PD and cancer therapy.

Ubiquitin C-terminal hydrolase-L1 (UCH-L1) is an unusual enzyme in that, in vitro, it catalyzes both amide hydrolysis and amide formation at the C-terminus of ubiquitin (Liu, Y., L. Fallon et al. (2002) Cell 111(2): 209-18). The dual hydrolase/ligase activity of UCH-L1, which is normally expressed only in brain and in gonads, is not a property of the systemically expressed homolog, UCH-L3. The molecular “switch” between the hydrolase and ligase activities is UCH-L1 dimerization. Importantly, the ubiquityl ligase activity expressed by UCH-L1 in vitro does not produce the K48 linkage that is required for proteasomal degradation (Pickart, C. M. (1997) Faseb J 11(13): 1055-66).

Whether the UCH-L1 hydrolase/ligase activity switch is operative in vivo is unclear, but several observations suggest that it may be. First, a UCH-L1 polymorphism that affects in vitro ligase but not hydrolase activity is linked to PD susceptibility (Maraganore, D. M., M. J. Farrer et al. (1999) Neurology 53(8): 1858-60) (Elbaz, A., C. Levecque et al. (2003) Mov Disord 18(2): 130-7). The polymorphic form that “protects” against PD (S18Y) has significantly less ligase activity and dimerization tendency when compared to the high-risk polymorph (S18). Second, the S18 UCH-L1 causes the accumulation of α-synuclein in cell culture, an effect thought to be relevant to PD pathogenesis, since increased expression of -synuclein causes a familial form of PD (Singleton, A. B., M. Farrer et al. (2003) Science 302(5646): 841). Third, the UCH-L1 polymorphic risk factor and a risk factor linked to a polymorphism in the α-synuclein promoter are synergistic (Maraganore, D. M., M. J. Farrer et al. (2003) Mov Disord 18(6): 631-6). The link between UCH-L1 and protein accumulation contrasts with the link between expression of typical ubiquityl ligases and protein degradation, consistent with the chemical difference between the products. Thus, UCH-L1 ligase activity seems to oppose protein degradation, where typical ligase activity is required for ubiquitin-dependent protein degradation. It is important to note that small-molecule proteasome inhibitors, which also promote protein accumulation, can induce PD-like dopaminergic neurodegeneration in cell culture and in animals (McNaught, K. S., C. Mytilineou et al. (2002) J Neurochem 81:301-306) (McNaught, K. S., L. M. Bjorklund et al. (2002) Neuroreport 13(11): 1437-41).

In addition to its normal expression in neurons, UCH-L1 is expressed in some non-neuronal tumors, especially those of neuroendocrine origin. In several tumors, UCH-L1 expression level correlates to the tumor stage (Ovaa, Huib et al. (2003) PNAS, vol. 101: no.8). UCH-L1 expression has also been detected in the following cancers: colon, lung (small cell and non-small cell), B cell lymphoma, and cervical (papillomavirus-positive).

Inhibition of UCH-L1 expression, with RNAi, or its activity, with small-molecule inhibitors, promotes proliferation of lung tumor cells, demonstrating that UCH-L1 expression is a response to, and not a cause of, proliferation (Liu, Y., H. A. Lashuel et al. (2003) Chem Biol 10: 837-46). The mechanism of this antiproliferative activity is unknown, but the activity itself is not surprising, given that small-molecule proteasome inhibitors (e.g., Velcade) are anticancer compounds. A potential mechanism is suggested by the fact that UCH-L1 interacts with JAB1, a protein involved in the regulation of p27, which is an important cell cycle inhibitor (Caballero, O. L., V. Resto et al. (2002) Oncogene 21(19): 3003-10). Thus, UCH-L1 may influence cellular levels of p27. Whether this effect involves its ligase activity is not clear.

According to the invention, without wishing to be limited by theory, the antiproliferative activity of UCH-L1 and its anti-PD activity may be mediated by opposite effects on the same molecular event. First, PD is more prevalent in men, while lung cancer is more prevalent in women, after tobacco intake has been taken into account (Pope, Ashley et al. (1999) J Gend Specif Med 2(6):45-51) (Baldereschi; Di Carlo et al. (2000) Neurology 55(9): 1358-63). Second, lung cancer is less prevalent in PD populations than one would expect given the fact that smoking is a risk factor for lung cancer and is protective against PD (Vanacore, Spila-Alegiani et al. (1999) Neurology 52(2): 395-8). According to the invention, both activities of UCH-L1 depend on protein farnesylation and membrane anchoring, an effect that is known to promote protein dimerization. Inhibition of this modification with a farnesyl transferase inhibitor (FTI) reduces accumulation of α-synuclein in COS-7 cells and rescues dopaminergic SH—SY5Y cells from the toxicity of α-synuclein. Furthermore, the activity of such compounds against lung tumor cell lines was dependent on the expression of UCH-L1. Accordingly, the invention provides a simple genetic test for susceptibility to one or more FTIs. According to the invention, a cell that expresses no, or low levels of, UCH-L1 is susceptible to cytostatic and/or cytotoxic effects of FTIs (cytostatic at low doses, and both cytostatic and cytotoxic at high doses).

Methods for Identifying Cancers and Patients for FTI Therapy

According to the invention, the strength of the anti-proliferative effect of a FTI on a cancer cell is inversely related to the level of UCH-L1 expression in the cancer cell. In one aspect of the invention, the level of responsiveness of a cancer cell to a FTI is determined as a function of the level of UCH-L1 expression in the cell. In some embodiments, the amount of FTI to be administered to a patient is determined based on the level of UCH-L1. For example, a higher dosage of a FTI may be administered to a patient with a cancer expressing high levels of UCH-L1. In another aspect of the invention, the level of UCH-L1 expression is compared to a reference or threshold amount of UCH-L1 expression. In some embodiments, if the level of UCH-L1 expression is greater than a reference or threshold amount (e.g., about 10% greater, about 25% greater, about 50% greater, about 75% greater, or about 100% greater than the reference or threshold amount, or more than 2 fold, more than 3 fold, more than 4 fold, more than 5 fold, or more than 10 fold higher than the reference or threshold amount) then the patient is identified as not susceptible to treatment with an FTI alone and an alternative treatment may be recommended. The alternative treatment can include, but is not limited to, surgery, another form of chemotherapy, and treatment with a combination of an FTI and a UCH-L1 inhibitor. In some embodiments, if the level of UCH-L1 expression is lower than a reference or threshold amount (e.g., about 10% lower, about 25% lower, about 50% lower, about 75% lower, or about 100% lower than the reference or threshold amount, or more than 2 fold, more than 3 fold, more than 4 fold, more than 5 fold, or more than 10 fold lower than the reference or threshold amount) then the patient is identified as susceptible to treatment with an FTI alone, or in combination with another therapy such as surgery or chemotherapy.

In some embodiments, a reference or threshold amount can be an absolute amount of UCH-L1 based on a measurement and a comparison to a standard curve. In other embodiments, a reference or threshold amount can be an amount of UCHL-1 expressed in a reference cell line (e.g., H1299, SH—SY5Y, CoLo, H169, U1906, U1285, CasKi, C33A, SU.DHL-4, Namalwa or Ramos) grown in culture. In other embodiments, a reference or threshold amount can be an amount of UCHL-1 expressed in a reference biological sample such as a tissue biopsy or cell sample (e.g., blood, sputum or other cell-containing solid or liquid biological sample) obtained from a subject and optionally grown in culture. The reference biological sample can be a standard biological sample obtained from the subject or patient to be treated (e.g., tissue from a patient biopsy, preferably a biopsy of a similar tissue in which the cancer is present), a standard tissue obtained from a reference subject (the reference subject can be of the same or of a different species). In other embodiments, the reference amount can be an amount of a reference gene expression that is not UCH-L1 expression. Preferably, the amount of a second reference gene is assayed in the same sample in which the amount of UCH-L1 is assayed. However, in some embodiments, the amount of second reference gene expression can be assayed in one of the reference cell lines or biological samples described herein. In other embodiments, the reference or threshold amount can be an alternative standard amount or signal that provides a constant reference against which an expression level of UCH-L1 can be compared (especially if the assay for UCH-L1 expression is performed in a reproducible assay using a known amount of starting cancer sample and a standard amount of detection reagent(s) under standard conditions.

According to some embodiments of the invention, a UCH-L1 reference or threshold amount is an amount that is known (or is shown) to be an amount above which a cell (e.g., a cancer cell) is not responsive to FTI treatment. In other embodiments, a UCH-L1 reference or threshold amount is an amount that is known (or is shown) to be an amount below which a cell (e.g., a cancer cell) is responsive or susceptible to FTI treatment.

The amount of UCH-L1 in a diseased tissue is preferably measured in cancer tissue from a patient to be treated. In some embodiments, the amount is measured in vivo in a subject using one or more methods described herein and known in the art. In other embodiments, the amount of UCH-L1 is measured in a sample obtained from a subject suspected of having cancer or a patient diagnosed as having cancer. The sample can be a solid tissue biopsy or a biological fluid sample. The sample can contain essentially cancer cells. Alternatively, the sample can contain a mixture of cancer cells and non-cancer cells. The amount of UCH-L1 can be obtained directly or extrapolated using appropriate controls and/or standards. According to the invention, the amount of UCH-L1 can be measured as a protein amount, a nucleic acid amount (e.g., an mRNA amount) or a UCH-L1 activity amount.

Detecting and Measuring UCH-L1 Expression

Techniques and detection reagents useful for detecting protein expression are well known in the art. As used herein “expression” of a protein encompasses the presence of nucleic acid molecules, e.g., mRNA transcribed from a gene of interest, and the presence of the protein itself. Protein expression may be identified by a variety of techniques including, but not limited to, DNA-DNA or DNA-RNA hybridizations and protein bioassay or immunoassay techniques, including membrane, solution, or chip based technologies for the detection and/or quantification of nucleic acid or protein molecules.

Nucleic Acid Detection

The presence of nucleic acid molecules encoding UCH-L1 can be detected in a biological sample, a cell culture, or in vivo, using UCH-L1 detection reagents useful for DNA-DNA or DNA-RNA hybridization or amplification using probes or fragments of nucleic acid molecules encoding UCH-L1 or one or more fragments thereof. Nucleic acid amplification based assays involve the use of oligonucleotides or oligomers based on the sequence of a gene encoding UCH-L1 to detect the level of RNA encoding UCH-L1. Preferred oligonucleotides for amplification or hybridization are between about 10 and about 100 nucleotides long, and preferably between about 20 and about 80 nucleotides long, for example about 20, about 30, about 40, about 50, about 60, about 70 or about 80 nucleotides long. In some embodiments, one or more oligonucleotides are complementary to a UCH-L1 mRNA. According to the invention, oligonucleotides include natural and synthetic nucleic acid molecules and modified nucleic acids. The backbone can be phosphodiester, phosphorothioate, peptide-based (e.g., PNA) or any other backbone. The oligonucleotide preferably contains a length of sequence that is at least 80%, preferably more than 90%, preferably 100% identical to a UCH-L1 target sequence. This length can be about 5, 10, 15, 20, 25, 30, 35, 40, 45, 50 or more bases long. Useful target sequences include the UCH-L1 gene and mRNA sequences such as those provided in SEQ ID NO: 1 (human UCH-L1) and in SEQ ID NO: 3 (mouse UCH-L1) or one or more variants thereof. The target sequences can be the entire sequences shown in SEQ ID NOS: 1 and 3, or complementary sequences thereof, or fragments of any one of the above (e.g., corresponding to the oligonucleotides described above). Sequences from other species also can be used.

A wide variety of labels and conjugation techniques are known to those skilled in the art and may be used in various nucleic acid assays. Means for producing labeled hybridization or PCR probes for detecting sequences related to polynucleotides encoding UCH-L1 include, but are not limited to, oligo-labeling, nick translation, end-labeling, or PCR amplification using a labeled nucleotide. Alternatively, the sequences encoding UCH-L1, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Pharmacia & Upjohn (Kalamazoo, Minn.), Promega (Madison, Wis.), and U.S. Biochemical Corp. (Cleveland, Ohio). Suitable reporter molecules or labels which may be used for ease of detection include, but are not limited to, radionuclides, enzymes, fluorescent, chemiluminescent, or chromogenic agents, as well as substrates, cofactors, inhibitors, magnetic particles, and the like.

Protein Detection

The detection of UCH-L1 proteins can be accomplished by any of a number of methods using UCH-L1 detection reagents that bind to UCH-L1 protein or one or more fragments thereof (e.g., the human UCH-L1 peptide shown in SEQ ID NO: 2 or a variant thereof or a fragment of any of the above, the mouse UCH-L1 shown in SEQ ID NO: 4 or a variant thereof or a fragment of any of the above, or UCH-L1 peptides from other species). Preferred methods for the detection of UCH-L1 proteins can involve, for example, immunoassays wherein UCH-L1 proteins are detected by their interaction with an UCH-L1 specific antibody. Antibodies useful in the present invention can be used to quantitatively or qualitatively detect the presence of UCH-L1 or antigenic fragments thereof. In addition, reagents other than antibodies, such as, for example, polypeptides that bind specifically to UCH-L1 proteins can be used in assays to detect the level of UCH-L1 protein expression. Alternatively, detection of UCH-L1 proteins may be accomplished by detection and measurement of levels of biological properties associated with UCH-L1 proteins such as enzymatice activity using a fluorescence-based assay described in (Liu, Y., H. A. Lasuel, et al. (2003) Chem Biol 10: 837-46) (e.g., measurement of UCH-L1 hydrolase activity using ubiquitin conjugated to aminocoumarin substrate).

Immunoassays useful in the practice of the invention include but are not limited to assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays, protein A immunoassays, to name but a few. These assays can be performed under denaturing conditions, non-denaturing conditions, conditions that maintain intact cells, and conditions that result in cell lysis.

Accordingly, detection reagents for measuring UCH-L1 levels include but are not limited to 1) antibodies immunoreactive with UCH-L1 polypeptide or antigenic fragments thereof and 2) UCH-L1 protein or fragments thereof (preferably labeled). Other binding reagents such as anti-sense peptides, which bind to epitopes within the UCH-L1 protein are also useful. Useful detection reagents (including antibodies) can bind to an UCH-L1 antigen with a dissociation constant of between about 1 μM and about 1 nM (e.g., about 1 nM, about 10 nM, or about 100 nM). However, the dissociation constant can be lower or higher. In some embodiments, an antibody can have a dissociation constant lower than 1 nM.

Antibodies of the present invention include polyclonal and monoclonal antibodies, as well as antibody fragments and derivatives that contain the relevant antigen binding domain of the antibodies. Antibody preparations that consists essentially of pooled monoclonal antibodies with different epitopic specificities, as well as distinct monoclonal antibody preparations are publicly available e.g., antiPGP-9.5 rabbit polyclonal antibody (Chemicon). Antibody detection reagents include, but are not limited to, antibodies that bind to a denatured UCH-L1 protein or portion thereof and antibodies that bind to a folded UCH-L1 protein or portion thereof.

The term “antibody” as used in this invention includes intact molecules as well as fragments thereof, such as Fab, F(ab′)2, and Fv which are capable of binding the epitopic determinant. These antibody fragments retain some ability to selectively bind with its antigen or receptor and methods for making antibodies or fragments are known in the art. (See for example, Harlow and Lane, Antibodies: A Laboratory Manual, Cold Spring Harbor Laboratory, N.Y. (1988), incorporated herein by reference).

A variety of techniques for detecting and measuring the expression of UCH-L1, using either polyclonal or monoclonal antibodies specific for the protein, are known in the art. Examples of such techniques include, but are not limited to, enzyme-linked immunosorbent assays (ELISAs), radioimmunoassays (RIAs), “sandwich”-assays, and fluorescence activated cell sorting (FACS). These and other assays are well described in the art. (See, e.g., Hampton, R. et al. (1990) Serological Methods, a Laboratory Manual, APS Press, St Paul, Minn., Section IV; and Maddox, D. E. et al. (1983) J. Exp. Med. 158:1211-1216).

Immunoassay formats can employ labeled antibodies to facilitate detection. RIAs have the advantages of simplicity, sensitivity, and ease of use. Radioactive labels are of relatively small atomic dimension, and do not normally affect reaction kinetics. However, radioactive assays have several disadvantages including a short shelf-life due to radioactive decay, a requirement for special handling and disposal, and a requirement for complex and expensive analytical equipment. RIAs are described in Laboratory Techniques and Biochemistry in Molecular Biology, by Work, T. S., et al., North Holland Publishing Company, NY (1978), with particular reference to the chapter entitled “An Introduction to Radioimmune Assay and Related Techniques” by Chard, T., incorporated by reference herein.

ELISAs have the advantage that they can be conducted using inexpensive equipment, and with a myriad of different enzymes, such that a large number of detection strategies—colorimetric, pH, gas evolution, etc.—can be used to quantitate the assay. In addition, the enzyme reagents have relatively long shelf-lives, and lack the risk of radiation contamination that attends to RIA use. ELISAs are described in ELISA and Other Solid Phase Immunoassays (Kemeny, D. M. et al., Eds.), John Wiley & Sons, NY (1988), incorporated by reference herein. For these reasons, enzyme labels are particularly preferred for use in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.; Voller, A., et al., 1978, J. Clin. Pathol. 31:507-520; Butler, J. E., 1981, Meth. Enzymol. 73:482-523). The enzyme that is bound to the antibody will react with an appropriate substrate, preferably a chromogenic substrate, to produce a chemical moiety that can be detected, for example, by spectrophotometric, fluorimetric, or by visual means. Enzymes that can be used to detectably label the antibody include, but are not limited to, horseradish peroxidase and alkaline phosphatase. Detection can also be accomplished by colorimetric methods that employ a chromogenic substrate for the enzyme.

No single enzyme is ideal for use as a label in every conceivable immunometric assay. Instead, one must determine which enzyme is suitable for a particular assay system. Criteria important for the choice of enzymes are turnover number of the pure enzyme (the number of substrate molecules converted to product per enzyme site per unit of time), purity of the enzyme preparation, sensitivity of detection of its product, ease and speed of detection of the enzyme reaction, absence of interfering factors or of enzyme-like activity in the test fluid, stability of the enzyme and its conjugate, availability and cost of the enzyme and its conjugate, and the like. Examples of suitable enzymes include, but are not limited to, peroxidase, acetylcholine esterase, alpha-glycerol phosphate dehydrogenase, alkaline phosphatase, asparaginase, beta-galactosidase, catalase, delta-5-steroid isomerase, glucose oxidase, glucose-6-phosphate dehydrogenase, glucoamylase, glycoamylase, luciferase, malate dehydrogenase, peroxidase, ribonuclease, staphylococcal nuclease, triose phosphate isomerase, urease, yeast-alcohol dehydrogenase, etc. Peroxidase and urease are among the more preferred enzyme labels, particularly because of chromogenic pH indicators which make its activity readily visible to the naked eye.

In lieu of such enzyme labels, chemiluminescent, radioisotopic, or fluorescent. labels may be employed for either labeling antibodies immunoreactive with UCH-L1 or UCH-L1 protein itself (i.e. for competition assays in which labeled UCH-L1 is mixed with cell lysates) or peptides or other reagents that bind to UCH-L1. Examples of suitable radioisotopic labels include, but are not limited to, ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd, etc. Examples of suitable chemiluminescent labels include, but are not limited to, luminal labels, isoluminal labels, aromatic acridinium ester labels, imidazole labels, acridinium salt labels, oxalate ester labels, luciferin labels, aequorin labels, etc. Examples of suitable fluorescent labels include, but are not limited to, fluorescein labels, isothiocyanate labels, rhodamine labels, phycoerythrin labels, phycocyanin labels, allophycocyanin labels, o-phthaldehyde labels, fluorescamine labels, etc. Among the most commonly used fluorescent labeling compounds are fluorescein isothiocyanate, rhodamine, phycoerythrin and fluorescamine. Likewise, a bioluminescent compound may be used to label the UCH-L1 antibody. The presence of a bioluminescence protein is determined by detecting the presence of luminescence. Important bioluminescence compounds for purposes of labeling are luciferin, luciferase and aequorin.

In methods of the invention, a solid phase, e.g., a conventional ELISA plate, is coated with detection reagents such as anti-UCH-L1 antibodies. For antibodies, the solid phase is usually poly(vinyl-chloride), but may be other polymers such as cellulose, polyacrylamide, nylon, polystyrene or polypropylene. The solid supports may be in the form of tubes, beads, discs or micro plates, or any other surfaces suitable for conducting an assay.

In some embodiments of the invention, the levels of UCH-L1 proteins can be analyzed by two-dimensional gel electrophoresis. Methods of two-dimensional electrophoresis are known to those skilled in the art. Samples, such as cells samples or tissue samples, are loaded onto electrophoretic gels for isoelectric focusing separation in the first dimension which separates proteins based on charge. A number of first-dimension gel preparations may be utilized including tube gels for carrier ampholytes-based separations or gels strips for immobilized gradients based separations. After first-dimension separation, proteins are transferred onto the second dimension gel, following an equilibration procedure and separated using SDS PAGE to separate the proteins based on molecular weight. When comparing biological samples derived from different experimental conditions or subjects, multiple gels are prepared for the different samples (including samples from control conditions or subjects).

Following separation, the proteins are transferred from the two-dimensional gels onto membranes commonly used for Western blotting. The techniques of Western blotting and subsequent visualization of proteins are also well known in the art (Sambrook et al, “Molecular Cloning, A Laboratory Manual”, 2.sup.nd Edition, Volume 3, 1989, Cold Spring Harbor). Standard procedures may be used, or the procedures may be modified as known in the art for identification of proteins of particular types, such as highly basic or acidic, or lipid soluble, etc. (See for example, Ausubel, et al., 1999, Current Protocols in Molecular Biology, Wiley & Sons, Inc., N.Y.). In some embodiments, unlabeled antibodies that bind to the UCH-L1 proteins are utilized in an incubation step, as in the procedure for a Western blot analysis. A second, preferably labeled, antibody specific for the first antibody is used in the Western blot analysis to visualize proteins that reacted with the first antibody.

According to the invention, UCH-L1 is a useful biomarker for assaying the susceptibility of a diseased tissue to the antiproliferative effects of an FTI.

Therapeutic Applications

Cancer Therapies

Methods and compositions of the invention are particularly useful for identifying cancers that are susceptible to treatment with FTIs. Methods and compositions of the invention are particularly useful to treat cancers that have been identified as having low levels of UCH-L1 (e.g., below a threshold amount). Methods and compositions of the invention are particularly useful to treat patients with cancers that have been identified as having low levels of UCH-L1 (e.g., below a threshold amount). In some embodiments, the UCH-L1 assay is performed as part of the diagnostic and therapeutic treatment. In other embodiments, the UCH-L1 assay is performed independently of the therapeutic recommendation (e.g., at a remote location in a laboratory that is not associated with a physician) and the treatment or therapy recommendation is based on the information or knowledge concerning the UCH-L1 levels in the diseased tissue to be treated.

Similarly, methods of the invention are useful to identify or select cancers or patients for treatment with a therapeutic regimen that does not involve an FTI. As discussed above, the therapeutic recommendation can be based on information or knowledge of UCH-L1 levels above a reference or threshold amount.

Methods and compositions of the invention are particularly useful in connection with cancers of neurolgical origin and small-cell and non-small-cell lung cancer, papillomavirus-positive cervical cancer, B-cell lymphoma, colon, and chronic lymphocytic leukemia. Methods and compositions of the invention are also useful particularly useful in connection with other cancers that can have variable levels of UCH-L1 expression (for example pancreatic and colon cancer small-cell and non-small-cell lung cancer, papillomavirus-positive cervical cancer, B-cell lymphoma, colon, and chronic lymphocytic leukemia). However, the invention can be used in connection with any cancer, including but not limited to: biliary tract cancer; bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease and Paget's disease; liver cancer; lung cancer including small cell lung cancer and non-small cell lung cancer; lymphomas including Hodgkin's disease and lymphocytic lymphomas; neuroblastomas; oral cancer including squamous cell carcinoma; esophageal cancer; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor.

Accordingly, methods and compositions of the invention can be used in connection with any multicellular or vertebrate subject having cancer or suspected of having cancer. According to the invention, a subject is preferably a human subject. However, a patient can also be a mammalian patient including, but not limited to, a dog, cat, mouse, rat, goat, sheep, horse, cow, donkey, or pig. A subject is preferably a patient diagnosed with cancer. A patient can be diagnosed with cancer using any recognized diagnostic indicator including, but not limited to, physical symptoms, molecular markers, or imaging methods. A subject can also be a subject at risk of developing cancer (e.g., a subject that has been exposed to a carcinogen or other toxin, a subject with one or more genetic predispositions for cancer, a subject with symptoms of early cancer, or a subject that has been treated for cancer and is at risk of cancer recurrence or metastasis).

Preferably, methods and compositions of the invention are useful for diagnosing and treating cancers of neuronal origin. Examples of cancers of neuronal origin include, but are not limited to, small-cell and non-small-cell lung carcinoma as well as papillomavirus-positive cervical cancer, B-cell lymphoma, colon, and chronic lymphocytic leukemia.

Therapeutic Compounds and Combinations

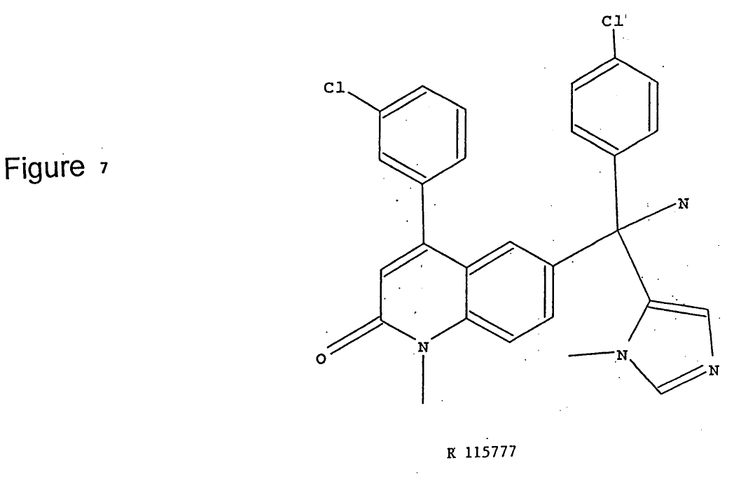

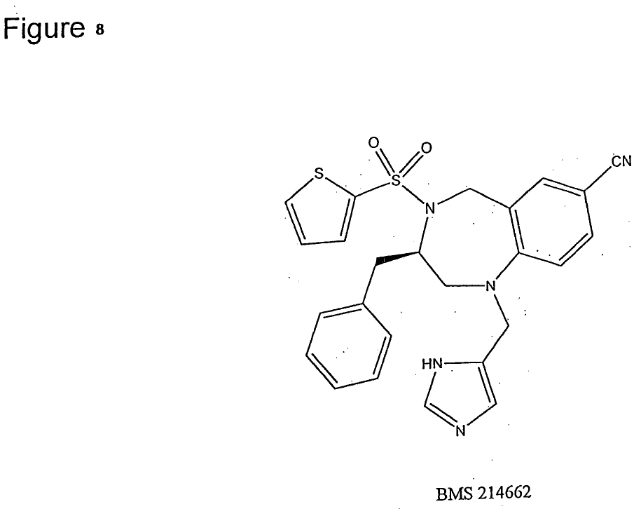

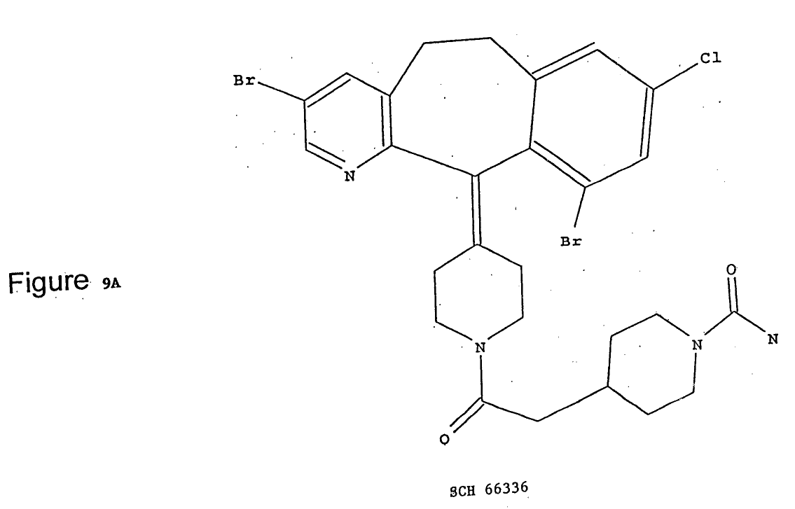

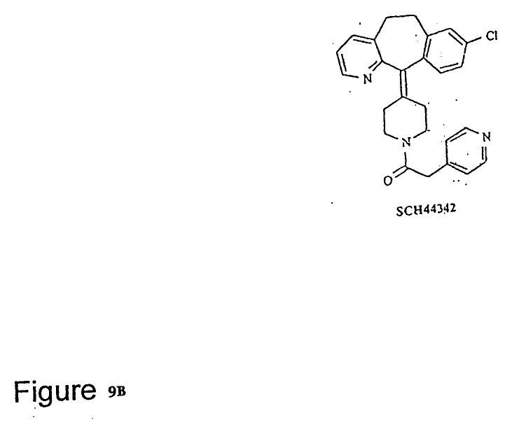

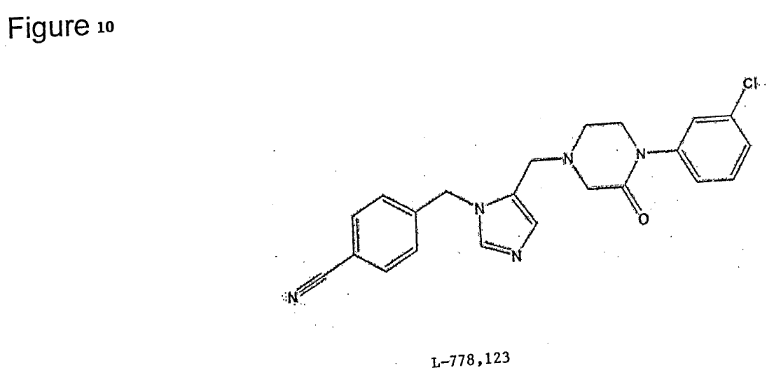

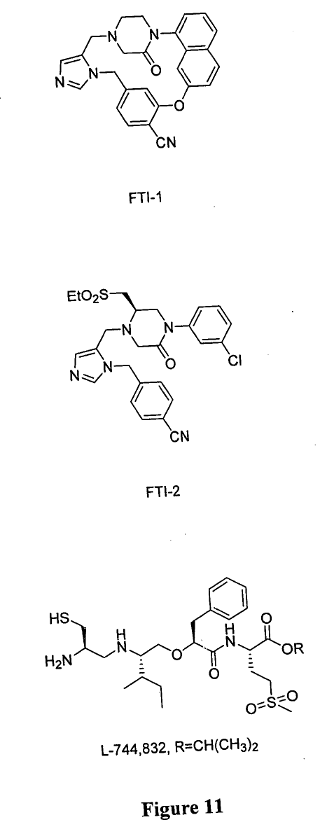

According to the invention, useful FTIs include any one or more FTIs such as those shown in FIGS. 7-10 and/or stereoisomeric forms, or pharmaceutically acceptable acid or base addition salt forms thereof, in therapeutically effective amounts.

The following publications describe useful farnesyl transefase inhibitor compounds and compositions and related synthetic and therapeutic methods: U.S. Pat. No. 6,545,020, U.S. Pat. No. 6,458,800, U.S. Pat. No. 6,451,812, U.S. Pat. No. 6,420,387, U.S. Pat. No. 6,187,786, U.S. Pat. No. 6,177,432, U.S. Pat. No. 6,169,096, U.S. Pat. No. 6,037,350 and U.S. Pat. No. 5,968,952 and WO 2002085364, WO 2002064142, WO 2002043733, WO 2001064252, US 2003212008, WO 2001064246, US 2003022918, WO 2001064226, US 2003027808, WO 2001064218, US 2003125326, WO 2001064217, US 2003078281, WO 2001064199, US 2003181473, WO 2001064198, US 2003050323, WO 2001064197, US 2003125268, WO 2001064196, US 2003060480, WO 2001064195, US 2003186925, WO 2001064194, US 2003100553, WO 2001062234, US 2003060450, WO 2001056552, US 2003027839, WO 2000001411, U.S. Pat. No. 6,545,020, WO 2000001386, U.S. Pat. No. 6,451,812, WO 9855124, U.S. Pat. No. 6,365,600, US 2002091138, WO 9721701, U.S. Pat. No. 6,169,096, U.S. Pat. No. 6,420,387, WO 2002024687, US 2003199547, WO 2002024686, US 2003207887, WO 2002024683, WO 2002072574, U.S. Pat. No. 6,358,961, and WO 03/080058; and WO 2003041658, WO 2002085819, WO 2001072721, WO 2000042849, WO 2003076660, WO 2002080895, WO 2002072085, WO 2002056884, WO 9730992, WO 9901434, US 2003162965, US 2002169313, US 2002002162, U.S. Pat. No. 6,537,988, US 2003134846, US 2003073677, US 2003092705, U.S. Pat. No. 6,645,966, U.S. Pat. No. 6,011,029, U.S. Pat. No. 6,387,926, U.S. Pat. No. 6,602,883, U.S. Pat. No. 6,455,523, and U.S. Pat. No. 5,925,757, WO 9804549, WO 2003072549, WO 2003047586, U.S. Pat. No. 6,358,968, US 20022119981, WO 9857970, WO 9857962, WO 9857948, U.S. Pat. No. 5,719,148, WO 9630363, U.S. Pat. No. 6,576,639, U.S. Pat. No. 5,874,442, U.S. Pat. No. 6,143,758, U.S. Pat. No. 6,214,828, WO 9857959, WO 9723478, and WO 2003092671, WO 200307660, WO 2002028409, US 2002077301, WO 2001076693, WO 2001060815, US 2002052380, WO 2001060368, US 2002010184, WO 2001032149, WO 2001007437, WO 2001005430, US 2002136744, WO 2000070083, WO 2000059930, US 2003220241, WO 2000025789, WO 2000025788, U.S. Pat. No. 6,329,376, WO 2000016778, WO 2000016626, WO 2000001702, U.S. Pat. No. 6,562,823, WO 2000001691, WO 2000001678, U.S. Pat. No. 6,160,118, WO 9909985, U.S. Pat. No. 6,387,903, WO 9910525, WO 9910524, WO 9910523, U.S. Pat. No. 6,103,487, U.S. Pat. No. 5,859,012, WO 9900654, U.S. Pat. No. 6,060,038, U.S. Pat. No. 5,856,326, WO 9630343, WO 9854966, WO 9844797, WO 9745412, WO 9738664, WO 9736889, WO 9736888, U.S. Pat. No. 5,919,785, WO 9736587, WO 9630343. The disclosures of these and all patents, patent publications and scientific publications listed herein are incorporated by reference herein in their entirety.

In some embodiments, combination therapies include one or more FTIs in association with one or more UCH-L1 inhibitors. Preferred UCH-L1 inhibitors include O-acyl oximes, and those described in Liu et al., 2003, Chemistry and Biology (10) 837-846, the relevant disclosure of which is incorporated herein by reference.

Therapeutic Preparations

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. In certain embodiments, the present invention relates to a compound represented by any of the structures outlined herein, wherein the compound is a single stereoisomer.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g., functioning as anti-proliferative farnesyl transferase inhibitor compounds), wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants, which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover.

In another aspect, the present invention provides “pharmaceutically acceptable” compositions, which comprise a therapeutically effective amount of one or more of the compounds described herein, formulated together with one or more pharmaceutically acceptable carriers (additives) and/or diluents. As described in detail, the pharmaceutical compositions of the present invention may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As set out herein, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%.

In certain embodiments, a formulation of the present invention comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

In certain embodiments, a compound or pharmaceutical preparation is administered orally. In other embodiments, the compound or pharmaceutical preparation is administered intravenously. Alternative routs of administration include sublingual, intramuscular, and transdermal administrations.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required to achieve the desired therapeutic effect and then gradually increasing the dosage until the desired effect is achieved.

In some embodiments, a compound or pharmaceutical composition of the invention is provided to a synucleinopathic subject chronically. Chronic treatments include any form of repeated administration for an extended period of time, such as repeated administrations for one or more months, between a month and a year, one or more years, or longer. In many embodiments, a chronic treatment involves administering a compound or pharmaceutical composition of the invention repeatedly over the life of the synucleinopathic subject. Preferred chronic treatments involve regular administrations, for example one or more times a day, one or more times a week, or one or more times a month. In general, a suitable dose such as a daily dose of a compound of the invention will be that amount of the compound that is the lowest dose effective to produce a therapeutic effect. Such an effective dose will generally depend upon the factors described above. Generally doses of the compounds of this invention for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight. However, lower or higher doses can be used. In some embodiments, the dose administered to a subject may be modified as the physiology of the subject changes due to age, disease progression, weight, or other factors.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition) as described above.

The compounds according to the invention may be formulated for administration in any convenient way for use in human or veterinary medicine, by analogy with other pharmaceuticals.

According to the invention, compounds for treating tumors, cancers, or other malignant growths in the vertebrate brain or central nervous system (CNS) can be formulated to enhance delivery or accumulation across the bloob brain barrier (BBB). The vertebrate brain (and CNS) has a unique capillary system unlike that in any other organ in the body. The unique capillary system has morphologic characteristics which make up the blood-brain barrier (BBB). The blood-brain barrier acts as a system-wide cellular membrane that separates the brain interstitial space from the blood.

The unique morphologic characteristics of the brain capillaries that make up the BBB are: (a) epithelial-like high resistance tight junctions which literally cement all endothelia of brain capillaries together, and (b) scanty pinocytosis or transendothelial channels, which are abundant in endothelia of peripheral organs. Due to the unique characteristics of the blood-brain barrier, hydrophilic drugs and peptides that readily gain access to other tissues in the body are barred from entry into the brain or their rates of entry and/or accumulation in the brain are very low.

Various strategies have been developed for introducing those drugs into the brain which otherwise would not cross the blood-brain barrier. Widely used strategies involve invasive procedures where the drug is delivered directly into the brain. One such procedure is the implantation of a catheter into the ventricular system to bypass the blood-brain barrier and deliver the drug directly to the brain. These procedures have been used in the treatment of brain diseases which have a predilection for the meninges, e.g., leukemic involvement of the brain (U.S. Pat. No. 4,902,505, incorporated herein in its entirety by reference).

Although invasive procedures for the direct delivery of drugs to the brain ventricles have experienced some success, they are limited in that they may only distribute the drug to superficial areas of the brain tissues, and not to the structures deep within the brain. Further, the invasive procedures are potentially harmful to the patient.

Other approaches to circumventing the blood-brain barrier utilize pharmacologic-based procedures involving drug latentiation or the conversion of hydrophilic drugs into lipid-soluble drugs. The majority of the latentiation approaches involve blocking the hydroxyl, carboxyl and primary amine groups on the drug to make it more lipid-soluble and therefore more easily able to cross the blood-brain barrier.

Another approach to increasing the permeability of the BBB to drugs involves the intra-arterial infusion of hypertonic substances which transiently open the blood-brain barrier to allow passage of hydrophilic drugs. However, hypertonic substances are potentially toxic and may damage the blood-brain barrier.

Peptide compositions of the invention may be administered using chimeric peptides wherein the hydrophilic peptide drug is conjugated to a transportable peptide, capable of crossing the blood-brain barrier by transcytosis at a much higher rate than the hydrophilic peptides alone. Suitable transportable peptides include, but are not limited to, histone, insulin, transferrin, insulin-like growth factor I (IGF-I), insulin-like growth factor II (IGF-II), basic albumin and prolactin.

Antibodies are another method for delivery of compositions of the invention. For example, an antibody that is reactive with a transferrin receptor present on a brain capillary endothelial cell, can be conjugated to a neuropharmaceutical agent to produce an antibody-neuropharmaceutical agent conjugate (U.S. Pat. No. 5,004,697 incorporated herein in its entirety by reference). The method is conducted under conditions whereby the antibody binds to the transferrin receptor on the brain capillary endothelial cell and the neuropharmaceutical agent is transferred across the blood brain barrier in a pharmaceutically active form. The uptake or transport of antibodies into the brain can also be greatly increased by cationizing the antibodies to form cationized antibodies having an isoelectric point of between about 8.0 to 11.0 (U.S. Pat. No. 5,527,527 incorporated herein in its entirety by reference).

A ligand-neuropharmaceutical agent fusion protein is another method useful for delivery of compositions to a host (U.S. Pat. No. 5,977,307, incorporated herein in its entirety by reference). The ligand is reactive with a brain capillary endothelial cell receptor. The method is conducted under conditions whereby the ligand binds to the receptor on a brain capillary endothelial cell and the neuropharmaceutical agent is transferred across the blood brain barrier in a pharmaceutically active form. In some embodiments, a ligand-neuropharmaceutical agent fusion protein, which has both ligand binding and neuropharmaceutical characteristics, can be produced as a contiguous protein by using genetic engineering techniques. Gene constructs can be prepared comprising DNA encoding the ligand fused to DNA encoding the protein, polypeptide or peptide to be delivered across the blood brain barrier. The ligand coding sequence and the agent coding sequence are inserted in the expression vectors in a suitable manner for proper expression of the desired fusion protein. The gene fusion is expressed as a contiguous protein molecule containing both a ligand portion and a neuropharmaceutical agent portion.

The permeability of the blood brain barrier can be increased by administering a blood brain barrier agonist, for example bradykinin (U.S. Pat. No. 5,112,596 incorporated herein in its entirety by reference), or polypeptides called receptor mediated permeabilizers (RMP) (U.S. Pat. No. 5,268,164 incorporated herein in its entirety by reference). Exogenous molecules can be administered to the host's bloodstream parenterally by subcutaneous, intravenous or intramuscular injection or by absorption through a bodily tissue, such as the digestive tract, the respiratory system or the skin. The form in which the molecule is administered (e.g., capsule, tablet, solution, emulsion) depends, at least in part, on the route by which it is administered. The administration of the exogenous molecule to the host's bloodstream and the intravenous injection of the agonist of blood-brain barrier permeability can occur simultaneously or sequentially in time. For example, a therapeutic drug can be administered orally in tablet form while the intravenous administration of an agonist of blood-brain barrier permeability is given later (e.g., between 30 minutes later and several hours later). This allows time for the drug to be absorbed in the gastrointestinal tract and taken up by the bloodstream before the agonist is given to increase the permeability of the blood-brain barrier to the drug. On the other hand, an agonist of blood-brain barrier permeability (e.g., bradykinin) can be administered before or at the same time as an intravenous injection of a drug. Thus, the term “co administration” is used herein to mean that the agonist of blood-brain barrier and the exogenous molecule will be administered at times that will achieve significant concentrations in the blood for producing the simultaneous effects of increasing the permeability of the blood-brain barrier and allowing the maximum passage of the exogenous molecule from the blood to the cells of the central nervous system.

The administration of the agents of the present invention may be for either prophylactic or therapeutic purpose. When provided prophylactically, the agent is provided in advance of disease symptoms such as any cancer symptoms. The prophylactic administration of the agent serves to prevent or reduce the rate of onset of symptoms. When provided therapeutically, the agent is provided at (or shortly after) the onset of the appearance of symptoms of actual disease. In some embodiments, the therapeutic administration of the agent serves to reduce the severity and duration of the disease or symptoms thereof (such as pain).

Kits

Diagnostic and therapeutic methods and compositions of the invention are ideally suited for a kit format. For example, the present invention provides a compartmentalized kit to receive in close confinement, one or more containers which comprises: a) a first container comprising a UCH-L1 binding reagent; and b) one or more other containers comprising one or more of the following: wash reagents and reagents capable of detecting the presence of bound agents from the first container.

As used herein, a compartmentalized kit includes any kit in which reagents are contained in separate containers. Illustrative examples of such containers include, but are not limited to, small glass containers, plastic containers or strips of plastic or paper. Particularly preferred types of containers allow the skilled worker to efficiently transfer reagents from one compartment to another compartment such that the samples and reagents are not cross-contaminated and the agents or solutions of each container can be added in a quantitative fashion from one compartment to another. Such containers include, but are not limited to, a container which will accept the test sample, a container which contains one or more of the reagents of the present invention used in the assay, containers which contain wash reagents (such as phosphate buffered saline, Tris-buffers, etc.), and containers which contain the reagents used to detect the bound reagent.

The types of detection reagents that can be used in the above described kits include, but are not limited to, labeled secondary agents, or in the alternative, if the primary reagent is labeled, enzymatic or agent binding reagents which are capable of reacting with the labeled reagent. One skilled in the art will readily recognize that reagents of the present invention can be readily incorporated into one of the established kit formats which are well known in the art.

The assays described herein can be performed, for example, using pre-packaged kits comprising at least one UCH-L1 binding or detection reagent.

In some embodiments, a kit according to the invention includes components that detect and/or measure UCH-L1 antigens in the biological sample. For example, where UCH-L1 proteins are detected and/or measured by enzyme linked immunoabsorbent assay (ELISA), such components may include an antibody directed to epitopes of the UCH-L1 proteins which can be used to detect and/or quantitate the level of UCH-L1 expression in the sample. The antibody itself may be detectably labeled with a radioactive, flourescent, colorimetric or enzyme label. Alternatively, the kit may contain a labeled secondary antibody.

In other embodiments, a kit can include on or more FTIs and/or one or more UCH-L1 inhibitors along with instructions on therapeutic applications based on known or assayed UCH-L1 levels.

The function and advantage of these and other embodiments of the present invention will be more fully understood from the examples described below. The following examples are intended to illustrate the benefits of the present invention, but do not exemplify the full scope of the invention.

### EXAMPLES

Experimental Procedures

Tissue culture: All cell lines were obtained by ATCC. SH—SY5Y and Cos-7 were grown in 10% FBS DMEM (Sigma). Cells were split the day before experiments including transfection, metabolic labeling and drug treatment.

Proteins and antibodies: UCH-L1 variants were purified according to the published procedure. Synuclein antibody (SYN-1) was purchased from Signal Transduction Lab. Actin antibody and FLAG antibody (M2) were from Sigma. UCH-L1 antibody (anti-PGP 9.5) was from Chemicon.

Chemicals: FTI-277 and lactacystin was purchased from Calbiochem. Crosslinking reagent DE was from Pierce. DMEM and MEM were purchased from Gibco. All the other material was purchased from Sigma.

Plasmids: C220S cDNA was generated by PCR site-specific mutagenesis. For the PCR, the 5′ primer is uchforw (CTAAAGCTTATGCAGCTCAAGCCGATGGAG) (SEQ ID NO: 5), and 3′ primer is uchc220s (CTAAGACTCGAGTTAGGCTGCCT TGCTGAGAGC) (SEQ ID NO: 6). Wt UCH-L1 served as the template. The PCR fragment was inserted into pcDNA vector. For S18YC220S mutant, S18Y UCH-L1 served as the template in PCR. For the FLAG tagged UCH-L1, the 5′ primer is FLAGuchforw (CTAAAGCTTATGGACTACAAGGATGACGACGACAAAGAT GCAGCTCAAGCCGATGGAG) (SEQ ID NO: 7), and the 3′ primer is uchrev (ATCCTCGAGTTAGGCTGCCTTGACGAGAGC) (SEQ ID NO: 8). Wt UCH-L1 or C220S served as the template. PCR fragment was purified and inserted into pcDNA vector. For the FLAG tagged UCH-L3, the 5′ primer is L3HindIII (CTAAAGCTTATGGACTACAAGGATGACGACGACAAAGATGGAGGGTCAACGCTGG CTG) (SEQ ID NO: 9), the 3′primer is L3XhoISAA (ATCCTCGAGCTATGCT GCAGAAAGAGCAATCGCA) (SEQ ID NO: 10). For the UCH-L3 CKAA variant, the 5′ primer is L3 HindIII and the 3′ primer is L3XhoICKAA (ATCCTCGAGCTA TGCTGCCTTAGAAAGAGCAATCGCATTAAATC) (SEQ ID NO: 11). α-synuclein degradation assay: Liphitamine 2000 was used to transfect COS-7 cells according to the Invitrogen protocol. Transfected cells were cultured at 37° C. for 48 hours before being treated with 35 μM lactacystin or DMSO. After 24 hours of incubation, the cells were lysed with Tris buffer (50 mM Tris, 2% SDS, 0.1% NP-40), and subjected to SDS-PAGE, followed by quantitative Western blotting.

Salt and detergent treatment of SV fraction: SV fraction was prepared as describe elsewhere. SV was incubated with various salts at designed concentration for 30 minutes on ice, or 1% Triton X-100 or control without salts and detergent. Treated SV was pelleted at 100,000 g for 30 minutes. Supernatants and pellets were subjected to SDS-PAGE and Western blotting.

Membrane fractionation: Cells were harvested by scraping and washed with PBS. Cell pellet was suspended in lysis buffer (50 mM Tris-HCl, 1 mM EDTA) supplemented with protease inhibitor cocktail (Sigma) and homogenized by passing through 26G needles 10 times. Suspension was clarified by spinning at 600 g for 5 minutes. Clarified suspension was ultracentrifuged at 100,000 g for 2 hours and separated into membrane and cytosol. Membrane fraction was washed with washing buffer (50 mM Tris-HCl, 1 mM EDTA 1M NaCl), and pelleted each time with bench-top centrifuge.

2D electrophoresis: For the isolation of total cellular protein, cultured SH—SY5Y cells maintained as described above were rinsed with ice-cold PBS. Cells were lysed in 1 ml dSDS buffer (50 mM Tris-HCl, pH 8.0 0.1% SDS) supplemented with protease inhibitor cocktail. Lysates were boiled for 3 min, and were treated with Dnase and Rnase as described. Lysates were precipitated with ice-cold acetone for at least 2 hours, and pellets were resuspended in 2D sample buffer (8M urea, 0.5% CHAPS, 0.2% DTT, 0.5% IPG buffer, 0.002% bromophenol blue). 2D electrophoresis was carried out according to manufacture's protocol (Amersham Life Science). 7 cm pH 4-7 strips were used. For SH—SY5Y membrane fraction, culture SH—SY5Y cells were rinsed with cold PBS and harvested with lysis buffer (50 mM Tris-HCl, pH 8.0, 1 mM ZnAc2, 250 mM sucrose). Lysate was passed through 25 G needles for several times and spun at 1000 g for 5 min. Supernatant was centrifuged at 200,000 g for 2 hours. Pellet was extensively washed with lysis buffer and extracted with cold acetone. Pellet was resuspended in 2D sample buffer.

Viral Infection: Viral infection and MTT assay in SH—SY5Y cells: The viruses were amplified and purified according to the published procedure. SH—SY5Y cells were grown on 100 mm petri-dishes and induced with 100 nM RA for 3-5 days before the virus infection with M.I.O at 75. Viruses were diluted with DPBS to desired M.I.O. After four hours of incubation, 10 ml growth medium was added. On the second day, cells were splitted into 96-well plates and treated with compounds for next 48 hours. The growth medium in each well was replaced with growth medium with 5 ug/ml MTT. Medium was removed after three hours incubation, and 200 ul isopropyl (0.04N HCl) was added into each well. The signal was read at 570 nm.

Viable cell counting: At stated time poins, SH—SY5Y cells were trypsinized with 100 ul trypsin-EDTA for 1 minute and neutralized with 400 ul growth medium. Cell suspension was made up by mixing 0.2 ml of cells in growth medium, 0.3 ml of HBSS and 0.5 ml of 0.4% Trypan Blue solution. Viable cell numbers were counted by standard cell counting chamber.

Western Blotting: Following transfer of SDS gels onto NC membrane, all membranes were blocked with 5% non-fat milk in TBST (50 mM Tris-HCl pH7.4, 150 mM NaCl, 0.1% Tween 20), and incubated with primary antibody overnight with 1% BSA in TBST, washed three times with TBST, and incubated with horseradish peroxidase-conjugated secondary antibody for 1 hour (Promega). Bound antibodies were detected using enhanced chemiluminascence (NEM).

### Example 1

### UCH-L1 is Farnesylated In Vivo and in Cell Culture

The UCH-L1 sequence contains the sequence CXXX, a consensus farnesylation site, at its C-terminus. This sequence is not present in UCH-L3. The possibility that this sequence was modified in vivo was investigated. First, the chemical nature of the previously reported association of UCH-L1 and synaptic vesicles from rat brain was probed.

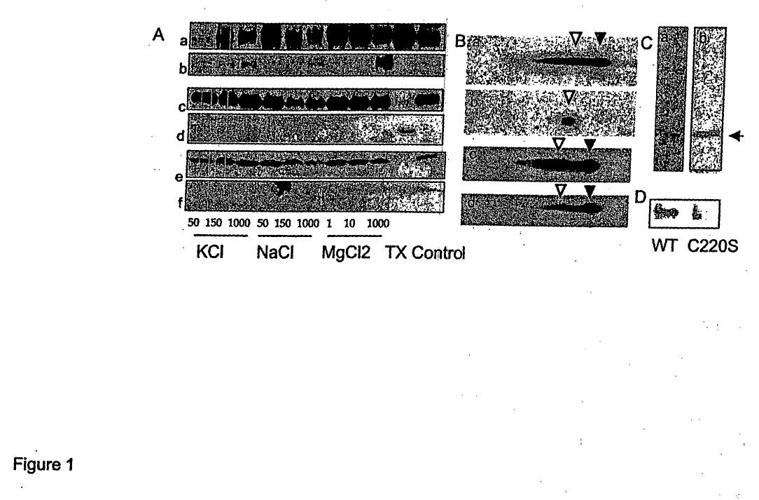

The results are shown in FIG. 1, panel (A): Effects of various amount of salt and non-ionic detergent on the dissociations of synapsin 1, synaphysin and UCH-L1 from SV was analyzed by treating aliquots of SV fraction with either KCl, NaCl, MgCl₂, or 1% Triton X-100. Membrane fraction and soluble fraction was separated by centrifugation and each fraction was subjected to SDS-PAGE followed by Western blots. a (synapsin I), c (synaphysin) and e (UCH-L1) are from pellet, and b (synapsin I), d (synaphysin) and f(UCH-L1) are supernatant fractions. Unlike synapsin (FIG. 1, panel A, rows a and b), which is not an integral membrane protein, and like synaptophysin (rows c and d), UCH-L1 (rows e and f) could not be separated from the vesicular fraction by increasing salt concentration. Only treatment with detergent was sufficient to solubilize UCH-L1, consistent with its farnesylation.

Analysis of various fractions from SH—SY5Y neuroblastoma cells (similar results from rat brain, not shown) by two-dimensional SDS-PAGE gel electrophoresis showed two major and two minor species in the total homogenate and one species in the membrane-associated fraction (FIG. 1 panel (B): More than 2 forms of UCH-L1 were present in SH—SY5Y cell (gel a) detected using 2D electrophoretic analysis followed by Western blotting. Only one of them (open arrow) is associated with membrane (gel b). Treatment of SH—SY5Y cells with FTI-277 (gel d) results in a significant decrease in the amount of membrane bound UCH-L1 (open arrow) without affecting the amount of cytosolic UCH-L1 (close arrow) when compared to cells treated with DMSO (gel c). This species was presumably the fully processed species: farnesylated, truncated and C-terminally methylated.

Consistent with this premise, treatment of the cells with the farnesyl transferase inhibitor FTI-277 decreased the amount of the membrane-associated species. In addition, a UCH-L1-containing species was immunoprecipitated from whole cell lysate by an anti-farnesyl antibody (Calbiochem). Finally, treatment of the cells with 14C-mevalonic acid or with 3H-farnesol resulted in incorporation of radiolabel into UCH-L1 (FIG. 1, panel (C)). UCH-L1 was modified with [¹⁴C] mevalonate (gel a) and [³H] farnesol (gel b) in vivo. (b). Transfection of the C220S mutant into COS-7 cells prevented radioincorporation and eliminated the membrane-associated species (not shown). FIG. 1, panel (D), shows that WT UCH-L1 but not the C220S variant was detected in the membrane fraction of COS-7 cells transfected with either of the UCH-L1 variants).

### Example 2

### Removal of the Farnesylation Site has No Effect on the In Vitro Enzymatic Activity or Aggregation Properties of UCH-L1

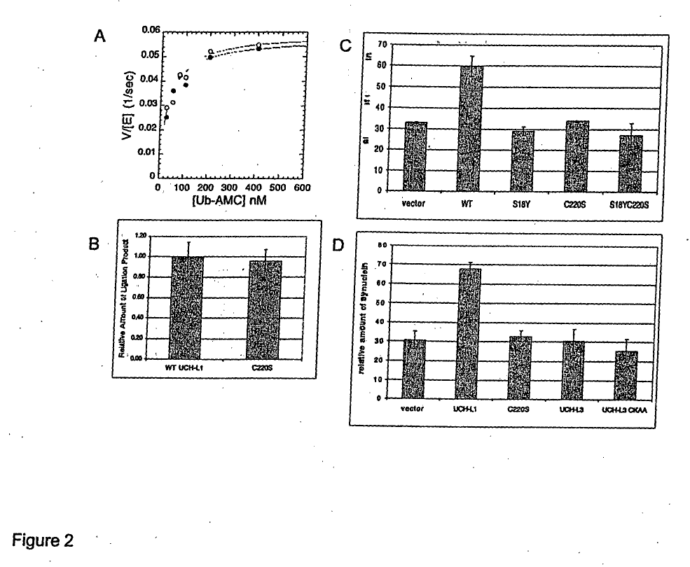

The C220S mutant as expressed in E. coli and purified using a published method. As expected from examination of structural models of UCH-L1, the point mutation had no effect on the in vitro hydrolase (FIG. 2, panel A) or ligase (panel B) activities. (A) Michaelis-Menten plot of various amount Ub-AMC titrated against either UCH-L1 WT (close circle) or C220S (open circle) showed comparable hydrolytic activities. (B) The mutation does not affect UCH-L1 in vitro ligase activity. In addition, the C220S mutation did not eliminate the propensity of S18 to oligomerize. This finding cleared the way to examine the effects of C220S in cell culture.

### Example 3

### Farnesylation and Membrane Association of UCH-L1 is Required to Promote Accumulation of a-synuclein in COS7 Cells

The C220S mutation eliminated the ability of S18 to promote α-synuclein accumulation in COS-7 cells but had no effect on the S18Y polymorph (FIG. 2, panel (C): the relative amount of 16 kDa α-synuclein was quantified and normalized against the amount of actin in transfected COS-7 cells with the presence of UCH-L1 variants. 100% accumulation of α-synuclein was achieved in cells treated with proteasome inhibitor lactacysteine). This finding suggested that farnesylation and membrane attachment of UCH-L1 are both required. In order to isolate the latter possibility, a mutant form of UCH-L3 was constructed in which the UCH-L1 farnesylation sequence was added to the UCH-L3 C-terminus. This protein did not cause accumulation of α-synuclein (panel (D) The relative amount of α-synuclein was compared among COS-7 cells transfected with UCH-L1 and UCH-L3 variants), although it was farnesylated and incorporated into the membrane (not shown). Thus, membrane attachment of an active hydrolase was insufficient to cause accumulation of α-synuclein.

### Example 4

### Inhibition of Farnesylation Rescues Cell Death Caused by a-synuclein Overexpression in SH—SY5Y Cells

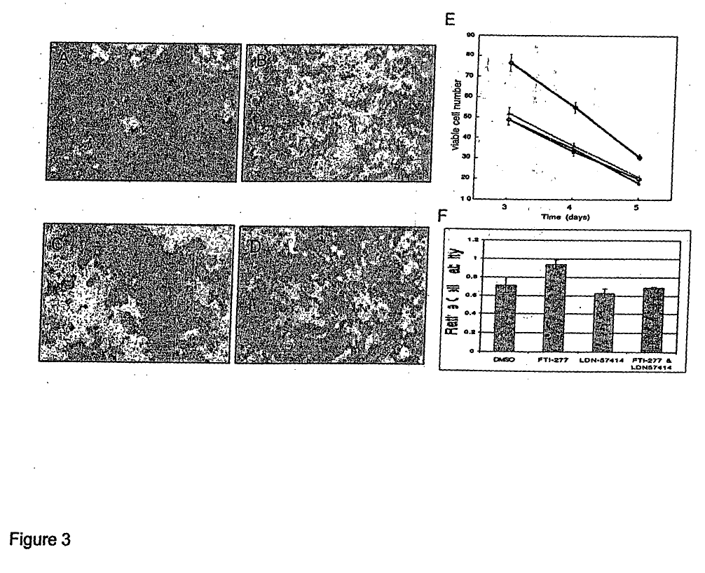

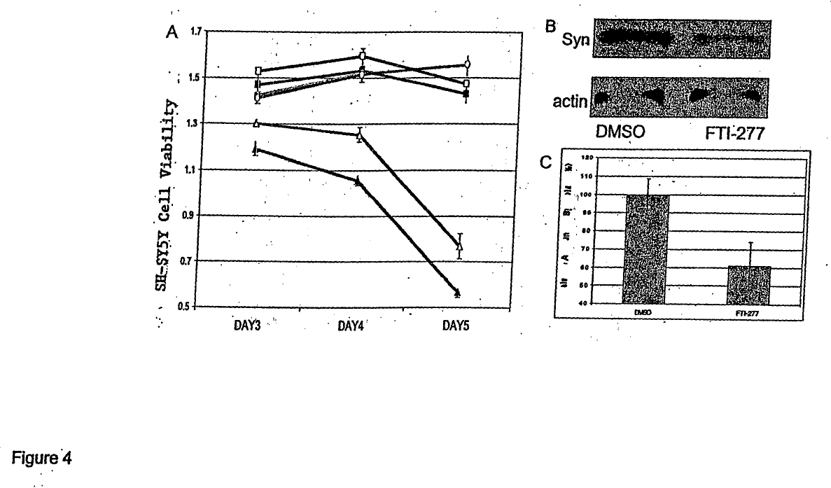

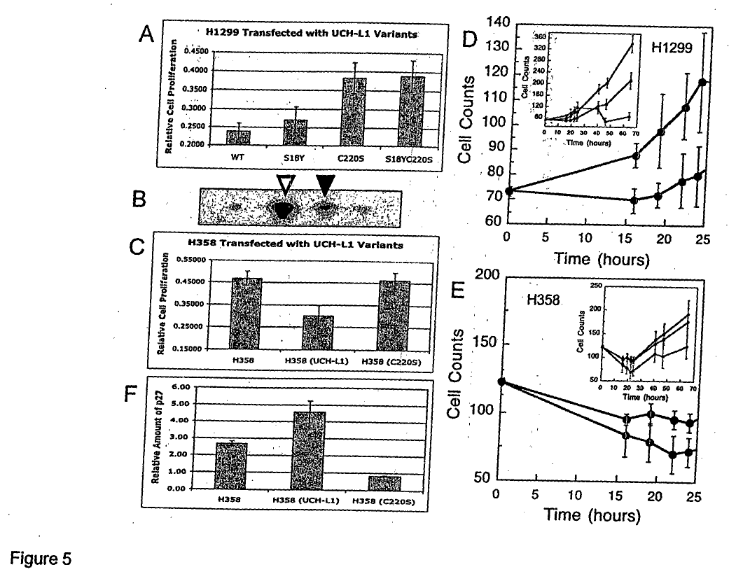

Since α-synuclein neurotoxicity is dose-dependent, it follows that accumulation of α-synuclein, caused by UCH-L1 farnesylation, should promote its toxicity. We demonstrated this to be true in mamallian neuroblastoma SH—SY5Y cells. This dopaminergic cell line has been used to demonstrate the rescue of α-synuclein toxicity by parkin, an effect that has also been demonstrated in primary dopaminergic cultures. These cells express high endogenous levels of UCH-L1. The α-synuclein gene was overexpressed (as compared to endogenous levels) via infection with an adenoviral vector and toxicity was demonstrated by the Trypan blue (FIG. 3) and MTT assays (FIG. 4). FIG. 3 shows SH—SY5Y cells infected by α-synuclein-expressing adenovirus treated with DMSO (A), FTI-277 (B), LDN57414 (C), FTI-277 and LDN57414 (D). (E) Viable cell numbers were quantified by counting the cells treated with either DMSO (red), FTI-277 (blue), LDN57414 (green) or LDN57414 and FTI-277 (black) that did not stain with trypan blue. The unit of y-axis is 10⁵/ml. (F) Cell viability was assessed by the amount of metabolic activity using MTT assay. FIG. 4 shows: (A) the viability of SH—SY5Y cells infected by α-synuclein-expressing adenovirus after treatment of DMSO or FTI-277, and of cells infected with lacZ-expressing adenovirus after treatment of DMSO or FTI-277, and of cells infected with empty adenovirus after treatment of DMSO or FTI-277 were assessed using MTT assay. The effect of FTI-277 on the α-synuclein accumulation in the SH—SY5Y infected with α-synuclein-expressing adenovirus were analyzed by Western blotting (B) and the amount of α-synuclein (C) was quantified using NIH Image program and normalized against the amount of actin.

The commercially-available small molecule farnesyl transferase inhibitor FTI-277, which had previously been shown to reduce the amount of membrane-associated, farnesylated species (FIG. 1, panel B, row d), resulted in a significantly decreased loss of cells (compare FIG. 3, panel B to panel A). This neuroprotective effect was eliminated by co-adminstration of the small-molecule UCH-L1 inhibitor (not shown), suggesting that the FTI effect was primarily due to its effect on UCH-L1. Treatment with FTI-277 reduced the total amount of UCH-L1 in SH—SY5Y cells and increased its rate of turnover (pulse-chase experiment not shown), in addition to reducing the amount of membrane-associated protein. This treatment also reduced the amount of α-synuclein in these cells (FIG. 4, panels B and C).

### Example 5

### Elimination of the UCH-L1 Farnesylation Site Eliminates Its Antiproliferative Activity in Lung Cancer Cells

Expression of UCH-L1 in non-small cell lung cancer (NSCLC) cell lines dramatically reduces cell proliferation FIG. 5, panel A)(Liu, Lashuel et al. 2003). This effect was dependent on UCH-L1 farnesylation; the C220S mutation eliminated the antiproliferative effect (panel A). Analysis of the H1299 cell line (and the A549 line, not shown) by 2D gel revealed that the farnesylated form in the predominant one in these cells. This difference between the NSCLC cells and the neuronal cells may explain why a significant difference in the effects of the two UCH-L1 polymorphs was not observed.

Even though there is no clinical study reporting UCH-L1 polymorphisms on the prevalence, incidence and mortality of lung cancer, our results would suggest that the variations at residue 18 have minimal impact on the progression of UCH-L1 positive lung tumor.

When H358 (a NSCLC that does not express UCH-L1) was stably transfected with UCH-L1 WT expression vector, the cell proliferation also decreased significantly when compared to the non-transfected H358 (FIG. 5A). Replacing WT with C220S UCH-L1 variant completely abolished this anti-proliferative effect, further supporting the hypothesis that this activity is mediated by the membrane-bound UCH-L1.

### Example 6

### Treatment of UCH-L1-Expressing Lung Cancer Cells with a Farnesyl Transferase Inhibitor Promotes Proliferation

To test the effect of farnesyl transferase inhibitor in a cell culture model, H1299 and H358 (FIG. 5B) were treated with 100 nM FTI-277 or DMSO. As a comparison, H1299 was also treated with 5 μM LDN-57414 (UCH-L1 inhibitor), which we have previously shown to increase H1299 cell proliferation (Liu et al 2003). Cell growth was quantified by counting the number of living cells as a function of time. While FTI-277 induced initial toxicity in H358 when compared to the DMSO control, the compound promoted initial cell growth in H1299 that is comparable to the pro-proliferative effect produced by treating the cell with LDN-57414 (FIG. 5B, Liu et al 2003). Interestingly, unlike LDN-57414 which continued to promote H1299 cell growth after 24 hours of compound treatment, FTI-277 began to show toxicity in H1299 cells, presumably due to the known inhibition on Ras farnesylation. The initial acceleration of cell growth in H1299, thus, may be the consequence of inhibition on UCH-L1 farnesylation. This possibility may explain the minimal clinical activity of farnesyl transferase inhibitor in treating patients with NSCLC.

### Example 7

### Farnesylation of UCH-L1 in Lung Cancer Cells Promotes Accumulation of the Tumor Suppressor p27

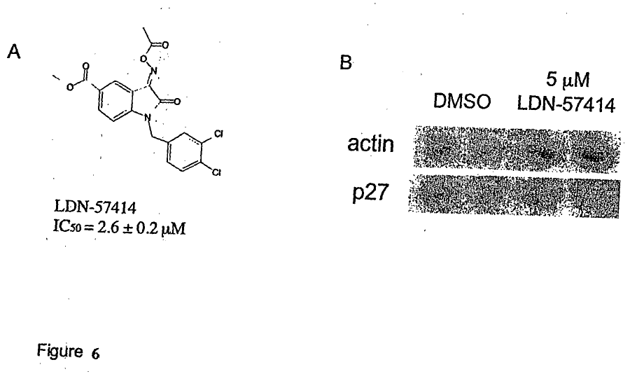

In α-synuclein cell model, we demonstrated that membrane bound UCH-L1 promoted accumulation of α-synuclein by inhibiting its degradation. To test if the enzyme activity of membrane bound UCH-L1 can cause similar dysfunction in proteasome degradation in lung cancer, the levels of the cyclin dependent kinase inhibitor p27 in H358 either transfected with UCH-L1 WT or C220S, or without transfection were quantified using western blot (FIG. 5C). p27 was previously shown to be a potential substrate of UCH-L1 in H1299 cell line (Caballero et al 2002) and its cellular level is regulated by proteasomal degradation pathway. The amount of p27 in cells transfected with WT UCH-L1 was significantly higher than that of non-transfected H358 or H358 transfected with C220S variant (FIG. 5C). H1299 treated with UCH-L1 inhibitor LDN-57414 also results in a decrease in amount of p27 when compared to the cells that was treated with DMSO (FIG. 6, panels A, B and C). Not only were these results consistent with the known role of p27 in cell proliferation and the observed cell growth rates among the different cell lines, they provided further support to our conclusion that the membrane bound UCH-L1's enzyme activity can result in inhibition of proteasomal degradation. And in good agreement with the notion that membrane association of UCH-L1 may promote enzyme dimerization thus the ligase activity and that the hydrolase activity is predominantly carried out in cytosol, the H358 transfected with C220S actually results in a decrease in the amount of p27 presumably through an enhancement of proteasomal degradation pathway. This difference in p27 levels between H358 non-transfected and transfected with C220S, however, did not correlate to effect on cell proliferation (FIG. 5A). This discrepancy may be caused by the presence of other cyclin dependent kinase inhibitors that also negatively regulate the cell cycle.

The following publications describe synthesis methods for farnesyl transefase inhibitor compounds and are incorporated herein by reference in their entirety U.S. Pat. No. 6,545,020, U.S. Pat. No. 6,458,800, U.S. Pat. No. 6,451,812, U.S. Pat. No. 6,420,387, U.S. Pat. No. 6,187,786, U.S. Pat. No. 6,177,432, U.S. Pat. No. 6,169,096, U.S. Pat. No. 6,037,350 and U.S. Pat. No. 5,968,952.

1. A screening method to identify cancers responsive to farnesyl transferase inhibitors, the method comprising the steps of:
detecting an amount of UCH-L1 in a cancer in a biological sample; and,
identifying the cancer as:
(i) a farnesyl transferase responsive cancer if the amount of UCH-L1 is below a threshold amount; or
(ii) a farnesyl transferase non-responsive cancer if the amount of UCH-L1 is above the threshold amount.

2. The method of claim 1, wherein the threshold amount of UCH-L1 is selected from the group consisting of an amount in a reference cell line, an amount in a reference cancer, and an amount that is twice that of normal tissue.

3. The method of claim 1, wherein the amount of UCH-L1 is detected using an assay selected from the group consisting of: an RNA expression assay, a protein expression assay, an ELISA, and a UCH-L1 activity assay.

4. The method of claim 1, wherein the biological sample is selected from the group consisting of: tissue biopsy, cell culture, and bodily fluids.

5. A method of treating cancer, the method comprising the steps of
identifying a cancer with a UCH-L1 amount below a threshold amount; and,
treating a subject having said cancer with a therapeutically effective amount of a farnesyl transferase inhibitor.

6. The method of claim 5, wherein the subject is a human.

7. The method of claim 5, wherein the farnesyl transferase inhibitor is selected from the group consisting of: SCH66336, L778123, BMS-214662, R115777 and FTI-277.

8. The method of claim 5, wherein two or more different farnesyl transferase inhibitors are administered and wherein said farnesyl transferase inhibitors are independently selected from the group consisting of: SCH66336, L778123, BMS-214662, R115777 and FTI-277.

9. The method of claim 5, wherein the farnesyl transferase inhibitor is administered in combination with another anticancer drug.

10. A screening method to identify cancers responsive to farnesyl transferase inhibitors, the method comprising the steps of:
detecting an amount of UCH-L1 in a cancer in a patient in vivo; and,
identifying the cancer as:
(i) a farnesyl transferase responsive cancer if the amount of UCH-L1 is below a threshold amount; or
(ii) a farnesyl transferase non-responsive cancer if the amount of UCH-L1 is above the threshold amount.

11.-16. (canceled)
